(19) United States
(12) Patent Application Publication
(10) Pub. No.: US 2008/0255589 A1
(43) Pub. Date: Oct. 16, 2008

(54) Multi-function clipping and harvesting device

(75) Inventors: Derek C. Blakeney, Saline, MI (US); Randal James Kadykowski, South Lyon, MI (US); David B. Maurer, Ann Arbor, MI (US)

Correspondence Address: TERUMO CARDIOVASCULAR SYSTEMS CORPORATION;ATTN: GAEL DIANE TISACK, 6200 JACKSON ROAD, ANN ARBOR, MI, 48103, US

(73) Assignee: Terumo Cardiovascular Systems Corporation

(21) Appl. No.: 11/786,302

(22) Filed: Apr. 11, 2007

(51) Int. Cl. A61B 17/122

(52) U.S. Cl. 606/142

(57) ABSTRACT

A multi-function minimally invasive vessel clipping and harvesting device includes at least two clipping devices positioned to advance surgical clips onto a tissue, and a severing device positioned to sever the tissue between the clips.

### CROSS-REFERENCE TO RELATED APPLICATIONS

Not applicable.

### STATEMENT REGARDING SPONSORED RESEARCH

Not applicable.

### BACKGROUND OF THE INVENTION

The present invention relates to the clipping, dissecting and harvesting of blood vessels.

The harvested vessels are used in many surgical procedures, including use as a coronary artery bypass graft, or in other cardiovascular procedures. As one example, in vascular and cardiovascular procedures, a blood vessel or vessel section, such as an artery or vein, is “harvested” (i.e., removed) from its natural location in a patient's body and is used elsewhere in the body. In coronary artery bypass grafting, for example, surgery, the harvested blood vessel is used to form a bypass between an arterial blood source and the coronary artery that is to be bypassed.

Endoscopic surgical procedures for harvesting a section of a blood vessel (e.g., the saphenous vein) subcutaneously have been developed in order to avoid disadvantages and potential complications of harvesting of the blood vessel in a more invasive fashion. In the past, the harvesting was done through a continuous incision (e.g., along the leg) that exposed the full length of the desired vein section. The continuous incision had been necessary in order to provide adequate exposure for visualizing the vein and for introducing the surgical instruments to dissect and harvest the vessel.

A more recent development has been a minimally-invasive technique that employs a small incision for locating the desired vessel and for introducing one or more endoscopic devices into the small incision.

Commercially available products for performing the endoscopic blood vessel harvesting procedure include a number of separate devices that are used. In the past, the clinicians have used one device to dissect the vessel from surrounding tissue. Another device is then typically used to cauterize and cut the branches from the dissected vessel while the vessel is still within the patient. In the past, the harvesting devices have included expensive radio frequency (RF) or ultrasonic generators that require additional support equipment and add additional time and difficulty to the harvesting procedure. Another device is often used to ligate and remove the vessel from the patient.

The cauterizing or sealing of the branch closed while still in the patient is generally viewed as an intermediate step that is adequate to seal the branches during vessel removal, but is not considered reliable enough to sustain the pressures once the harvested vessel is sutured to the heart as a long-term bypass graft. Therefore, once the harvested vessel is taken out of the patient's arm, another device has been used to securely seal the branch stubs on the harvested vessel. The branch stubs are generally sutured, tied or clipped closed as an additional measure of safety to assure that the branch stubs do not leak.

Since there is an increasing occurrence of minimally invasive surgery, however, there is also a growing need for more efficient and compact devices that shorten the time and invasiveness of the surgery.

It would be especially useful to have a device that performed multiple functions so that there would be fewer intrusions into the open wound in the patient.

### SUMMARY OF THE INVENTION

In one aspect, there is provided a multi-function device for harvesting a vessel post dissection. The multi-function device includes a sheath that is to be at least partially inserted in a body through a cut skin portion. First and second clipping devices are positioned within the sheath. Each clipping device advances a surgical clip and closes the advanced surgical clip around the vessel or branch. A severing device is also positioned within the sheath and is advanced to sever the clipped vessel or branch.

In certain aspects, the severing device is interposed between the first and second clipping devices. Also, a vision system positioned within the sheath aids in the visualization of the clipping and severing steps.

In another aspect, the sheath can include a dissector tip in an axial position over a distal end of the sheath. The dissector tip is movable between an open position and a closed position such that the first and second clipping devices are in an exposed position when the tip is in the open position.

The multi-function device includes a clip advancing mechanism having a movable housing to engage at least one of the first or second clipping devices. The movable housing includes a first opening through which a guide bar extends, and second opening through which a pivot bar extends. The movable housing can be slidably moved along a longitudinal axis A extending through the guide bar; slidably moved along a longitudinal axis B extending through the pivot bar; and/or pivotably moved about the longitudinal axis B through the pivot bar. The movable housing is configured to substantially activate: i) the first clipping device followed by the severing device; ii) the second clipping device followed by the severing device; or, iii) the first clipping device and the second clipping device followed by the severing device.

In yet another aspect, there is provided a method of severing branches and/or vessels during harvesting from a body. The method includes:

inserting a distal end of the multi-function device into the body alongside the vessel to form a cavity substantially surrounding the vessel or branch;

activating the first and second clipping devices to seal the vessel or branch; and

moving the severing device to a position substantially adjacent to the sealed vessel or branch, and activating the severing device, whereby the sealed vessel or branch is severed.

The multi-function device allows the clinician to have several different operating choices: i) the first and second clipping devices are actuated substantially simultaneously; ii) either the first or the second clipping devices are actuated sequentially; iii) same as i) but followed by the severing device; and, iv) same as ii) but followed by the severing device.

Also, in certain embodiments, the multi-function device further includes a light source and an imaging system and/or an insufflation device for supplying a gas subcutaneously to an area adjacent to the vessel to be dissected and harvested.

Various objects and advantages of this invention will become apparent to those skilled in the art from the following detailed description of the preferred embodiment, when read in light of the accompanying drawings.

### BRIEF DESCRIPTION OF THE DRAWINGS

FIG. 1 is a structure diagram, broken away and partially in phantom, showing a handle portion and a tip or distal end of one embodiment of a multi-function device.

FIG. 2 is a structure diagram, partially in phantom, showing an enlarged view of the distal end of the multi-function device.

FIG. 3 is a structure diagram of one embodiment, partially in phantom, of a portion of the distal end of the multi-function device.

FIG. 4 is a structure diagram of one embodiment of a distal end of a multi-function device and further having a dissecting tip in an open position.

FIG. 5A is a structure diagram, in a perspective view, showing one embodiment of an engagement mechanism for the clipping devices.

FIG. 5B is a structure diagram, in a side elevational view, of the embodiment shown in FIG. 5A.

FIG. 6A is a structure diagram, shown in a top view, of the embodiment of FIG. 5A where a first clipping device is being contacted.

FIG. 6B is a structure diagram, shown in a top view, of the embodiment of FIG. 5A where a second clipping device is being contacted.

FIG. 6C is a structure diagram, shown in a top view, of the embodiment of FIG. 5A where first and second clipping devices are being contacted.

FIG. 7A is a structure diagram, shown in an end elevational view, of the embodiment of FIG. 5A where a first clipping device is being contacted.

FIG. 7B is a structure diagram, shown in an end elevational view, of the embodiment of FIG. 5A where a second clipping device is being contacted.

FIG. 7C is a structure diagram, shown in an end elevational view, of the embodiment of FIG. 5A where first and second clipping devices are being contacted.

FIG. 8 is a structure diagram, in a side elevational view, of another embodiment of an engagement mechanism for the clipping devices and for the severing device.

FIG. 9A is a structure diagram, shown in an end elevational view, of the embodiment of FIG. 8 where a first clipping device and the severing device are being contacted.

FIG. 9B is a structure diagram, shown in an end elevational view, of the embodiment of FIG. 8 where a second clipping device and the severing device are being contacted.

FIG. 9C is a structure diagram, shown in an end elevational view, of the embodiment of FIG. 8 where first and second clipping devices and the severing device are being contacted.

### DETAILED DESCRIPTION OF THE PREFERRED EMBODIMENT(S)

There is provided herein a multi-function device 10 and a system for using such device. The multi-function device is useful for clipping the branches closed and then cutting the clipped branches during the actual harvesting procedure. The multi-function device 10 provides a less expensive and faster system for the harvesting of a vessel. In one particular aspect, the multi-function device 10 eliminates the need for a subsequent tying or clipping procedure of the harvested vessel once the vessel is outside the body. The multi-function device 10 also decreases the amount of time the harvested vessel is outside the patient's body.

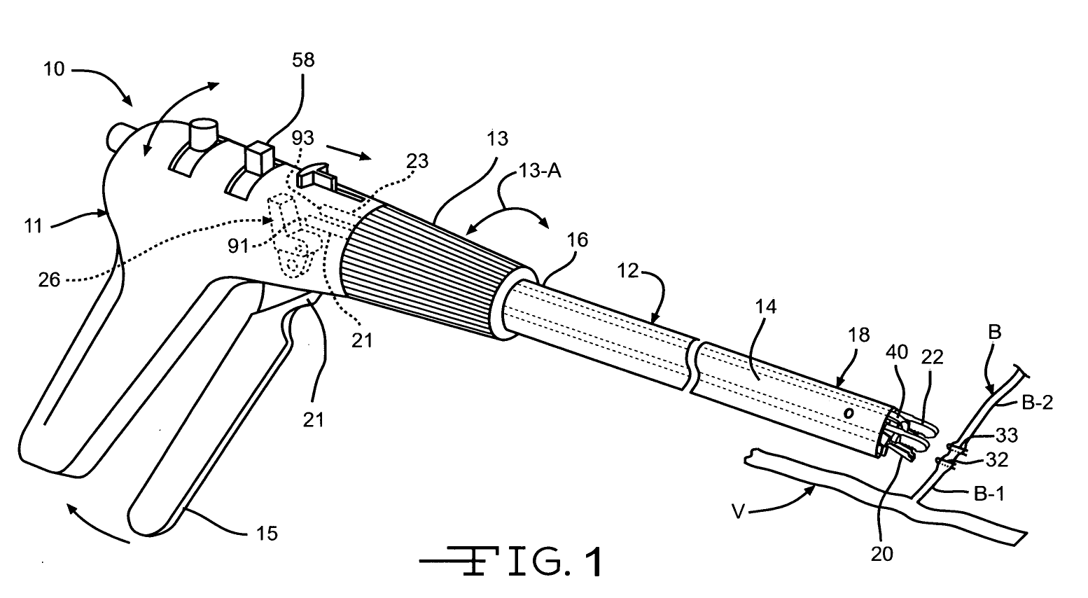

Referring now to FIG. 1, one embodiment of a multi-function device 10 for harvesting a vessel V and for severing branches B from the vessel V is schematically illustrated.

The multi-function device 10 includes a handle 11 and an elongated sheath 12. The sheath 12 defines an inner space 14 and has a proximal end 16 and a distal end 18. The multi-function device 10 includes first and second clipping devices 20 and 22 that are axially positioned within the sheath 12.

In certain embodiments, the handle 11 can include an orientation member 13 that allows rod portion of sheath 12 to be rotated about its axis, as shown by arrow 13-A, so that the clinician can maneuver the distal end 18 to the desired position within the patient.

The first and second clipping devices 20 and 22 generally have first and second proximal ends 21 and 23, respectively, that are mounted in the handle 11, as further explained below.

The first and second clipping devices 20 and 22 are axially aligned in a parallel relationship within the sheath 12. The first clipping device 20 is configured to apply a first surgical clip 32 and to close the surgical clip 32 around a selected portion of the branch B or vessel V. Similarly, the second clipping device 22 is configured to apply a second surgical clip 33 and to close the surgical clip 33 around an adjacent selected portion of the branch B or vessel V.

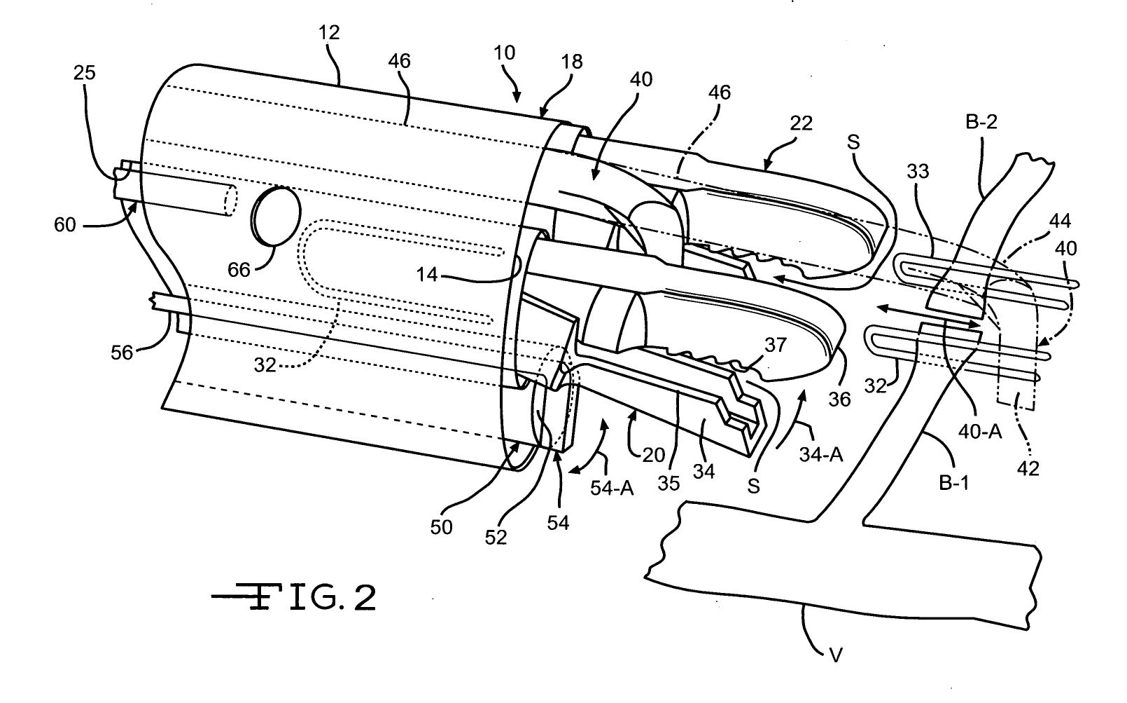

Referring now to FIGS. 1 and 2 in particular, the first and second clipping devices 20 and 22 will be described in detail. In the embodiment shown, each clipping device 20 and 22 has the same configuration. It is to be understood, however, that in certain embodiments, it may be desired that the clipping devices 20 and 22 can each have a configuration to deliver different types of clips. For ease of illustration herein, however, the first and second clipping devices 20 and 22 are shown herein as having the same configuration, and only one will be described in detail.

It should be noted, however, that non-limiting examples of suitable clipping devices include the Ligaclip MCA® and Ligaclip Allport® made by Ethicon Endo-Surgery Inc. of Cincinnati, Ohio, the Weck® Hemoclip® made by Teleflex of Research Triangle Park, N.C., and the Surgiclip® AutoSuture made by United States Surgical of Norwalk, Conn. It is to be understood that there are different mechanisms for the application of the clips within the clipping devices and that such mechanisms can be used with the embodiments described herein. As such, while various types of clipping devices are useful in the multi-function device 10, illustrated herein is one such suitable clipping device where the clipping device 20 generally includes a clip dispensing mechanism 26.

The clip dispensing mechanism 26 is configured to advance the surgical clip 32 along a cartridge or channel 25 that holds a plurality of clips. The dispensing mechanism 26 is configured to receive and position the surgical clip 32 around a vessel V or branch B.

During use of the multi-function device 10, the distal end 18 of the sheath 12 is positioned adjacent the vessel V or branch B. The clip dispensing mechanism 26 receives the advancing surgical clip 32, at least momentarily holds the clip 32 in a ready position, and then pinches or closes the clip 32 about the branch B.

In the embodiment shown in the FIGURES, the clip dispensing mechanism 26 includes opposing first and second sealing members 34 and 36. The first sealing member 34 is configured to be moved between open and closed positions as shown by arrow 34-A. In use, when in an open position, a space S is defined between the first and second sealing members 34 and 36. The first and second sealing members 34 and 36 are positioned around portions of the branch B. Once that portion of the branch B is in the space S between the first and second sealing members 34 and 36, the clip dispensing mechanism 26 is actuated, thereby delivering a clip 32 to seal the branch B. As shown in FIGS. 1 and 2, the first and second clipping devices 20 and 22 are activated to dispense separate surgical clips 32 and 33 onto separate portions B-1 and B-2 of the branch B.

In certain embodiments, one or more of the sealing members 34 and 36 can have engaging faces 35 and 37, respectively, that are configured to aid in securing the branch to prevent the branch from slipping during the positioning of the clip 32 in the clipping step.

In certain embodiments, the first and second clipping devices 20 and 22 are positioned within the sheath 12 such that the first and second clips 32 and 33 are separated by about a centimeter or less on the branch B. For example, in the use of such embodiments, the first clip 32 can be applied at a portion of the branch B that is right next to the main vessel V (i.e., the graft vessel side), and the second clip 33 can be applied roughly a centimeter or less further down the branch (i.e., the patient side). Also, during certain procedures, it is desired that the first, or graft-side, clip 32 be positioned as close as possible to the main vessel V. The close positioning of the surgical clip 32 to the longitudinal axis of the harvested vessel V is done in order to provide a smooth and continuous flow path in the main vessel's inner diameter, thereby avoiding flow turbulence once the vessel has been grafted into place.

In one particular embodiment, first and second clipping devices 20 and 22 are engaged substantially simultaneously so that two clips 32 and 33 are applied in a single actuation step.

As best seen in FIGS. 2 and 3, the multi-function device 10 includes a severing device 40 that is also axially positioned within the sheath 12. It is to be understood that various types of severing devices can be used in the multi-function device 10 and that non-limiting examples of suitable severing devices include a knife blade, as illustrated herein, a radio-frequency powered bovie, an ultrasonic cauterizing tool, or a bipolar electrocautery tool.

In the embodiment shown in the FIGURES herein, the severing device 40 includes a cutting tool 42 on a distal end 44 of an axially extending rod 46. The severing device 40 is activated by axially extending cutting tool 42 as shown by arrow 40-A. In use, the cutting tool 42 is maneuvered adjacent to the captured branch portions B-1 and B-2. The cutting tool 42 is then advanced in a direction between the first and second surgical clips 32 and 33.

In certain embodiments, the severing device 40 can be actuated just after the surgical clips 32 and 33 are applied. It is also within the contemplated scope of the system described herein that the multi-function device 10 can also be used to clip the patient sides of the main vessel once the vessel branches have bee sealed, and then cut the ends of the main vessel. This procedure is colloquially called a “stab and grab” phase of the harvesting procedure. In such uses, the first and second clipping devices provide additional gripping and holding of the vessel being grabbed and removed and only the patient-side clipping device is actuated. (The ends of the graft should remain unobstructed).

It is also contemplated that the multi-function device 10 can be used in a suturing manner by the clinician. In such procedures, the multi-function device 10 can be used after harvesting a graft from the patient. The harvested graft can be further clipped or tied (sutured) to close the graft's branch stubs even if they have been sealed during harvesting via electrocautery, ultrasonics, etc., as a further precaution against leaks or ruptures of the harvested vessel.

Also, in certain situations, when the harvested vessel is examined by the clinician, there can be a determination that the harvested vessel should be modified for final use in the grafting procedure. In such situations, the multi-function device can be again used to dispense a surgical clip close to the patient side of the vessel and to further trim any excess branch portion from the vessel.

Referring again to the FIGURES, and in particular to FIGS. 2 and 3, in certain embodiments, the multi-function device 10 includes a vision system 50. In the embodiment shown, the vision system 50 is illustrated as an endoscope, as currently utilized in Terumo CVS's Virtuosaph® endoscope device. In such embodiment, the endoscope is hard-wired to a video output monitor (not shown). In other embodiments, the vision system 50 can be a wireless camera sensor sometimes also referred to as “chip on the tip” technology. In such embodiment, the wireless vision system helps preserve valuable space within the sheath 12.

In the embodiment shown in FIGS. 2 and 3, the imaging system 50 is positioned within the sheath 12. The imaging system 50 has a lens 52 and a suitable image-receiving device (not shown) that converts images into signals for transmission, recording and/or storage, and/or takes photographs of such images. During use, the imaging system 50 is operated so that an image beyond the distal end 18 of the sheath 12 is visible when the branch B is being clipped and severed from the vessel V.

In certain embodiments, the vision system 50 can also include a wiper 54 that is positioned on a wiper rod 58 (shown in FIG. 1). The wiper rod 56 is axially positioned in the sheath 12 and is connected to a wiper switch 56. The wiper rod 56 with the wiper 54 attached thereto extends from the distal end 18 of the sheath 12. The wiper 54 is adjacent to the lens 52 and is at least partially pivotable about a longitudinal axis extending through the rod 56 (as shown by arrow 54-A in FIG. 2). The wiper 54 can be pivotably moved across the lens 52 when needed in order to clear any fluids or debris away from the lens 52.

In certain embodiments, the multi-function device 10 further includes an insufflation device 60. The insufflation device 60 is configured to supply a gas subcutaneously to an area adjacent to the vessel or branch. The insufflation device 60 can be axially positioned within the inner space 14 of the sheath 12. The insufflation device 60 includes a supply (not shown) of a suitable gas that is supplied into the sheath 12. In certain embodiments, as shown in FIGS. 2 and 3, the insufflation device 60 can have a supply line 62 having a discharge end 64. In certain embodiments, the discharge end 64 of the supply line 62 is located near the distal end 18 of the sheath 12. The sheath 12 can include one or more discharge ports or holes 66 that are in communication with the inner space 14. The gas escaping from the ports 66 enters the patient and keeps the surrounding tissue away from the vessel and branch.

Also, in certain embodiments, as shown in FIG. 4, the multi-function device 10 can further include a dissector tip 80 that is axially positioned over the distal end 18 of the sheath 12. At least a portion of the dissector tip 80 is transparent in order to allow visualization of the vessel V and the surrounding tissue, as further explained below. As best seen in FIG. 4, the dissector tip 80 can include two or more dissecting tip members 82 and 84 that define an inner space 85. The dissecting tip members 82 and 84 are movable between an open position and a closed position, as shown by arrow 82-A. When the dissector tip 80 is in the open position the first and second clipping devices 20 and 22 are in an exposed position.

At least one of the tip members 82 and/or 84 can have a conical or other tapered shape to aid in the harvesting of a vessel.

To begin the dissection procedure, the dissector tip 80 is inserted through an initial incision in the patient. In operation, the dissector tip 80 is pressed into the tissues surrounding the vessel, thereby forming a tunnel or cavity around the vessel. In certain embodiments, it is desired that the dissector tip 80 be pressed into the surrounding tissue generally along the direction of the vessel in order to separate the vessel from adjacent tissue without damage to the vessel or the surrounding tissue.

Upon inserting the multi-function device 10 under the patient's skin, it is possible to obtain an image illuminated by the vision system 50. The dissector tip 80 is used to perform an initial, or blunt, dissection of the vessel from the surrounding tissue. The tip members 82 and 84 are then moved to the open position, as illustrated in FIG. 4.

The first and second clipping devices 20 and 22 are activated to seal the branch portions B-1 and/or B-2. The severing device 40 is moved to a position substantially adjacent to the sealed branch portions B-1 and B-2. The severing device 40 is activated, thereby severing the branch B from the vessel V.

It is also within the contemplated scope of the various embodiments described herein that one or more of the clipping devices 20 and 22 can have a two-step firing procedure. In such embodiments, at least one or more of the first and second clipping devices 20 and 22 can be advanced to a gripping position adjacent to the vessel or branch to be sealed and cut. In a first step, the branch is first grabbed, and optionally, at least temporarily constricted. In a second step, a final decision is made to proceed with the actual clipping of the branch B and the clip is delivered to seal the branch.

Referring now to FIGS. 1 and 5-7, one embodiment of a suitable clip dispensing mechanism 26 is schematically illustrated. The clip dispensing mechanism 26 is enclosed within the handle 11. The clip dispensing mechanism 26 is positioned adjacent to the proximal ends 21 and 23 of the clipping devices 20 and 22, respectively.

In the embodiment shown, a first spring 90 is axially positioned over the first proximal end 21 of the first clipping device 20. The first proximal end 21 terminates at a first flange 91, against which the first spring 90 is biased. Similarly, a second spring 92 is axially positioned over the second proximal end 23 of the second clipping device 22. The second proximal end 21 terminates at a second flange 93, against which the second spring 92 is biased.

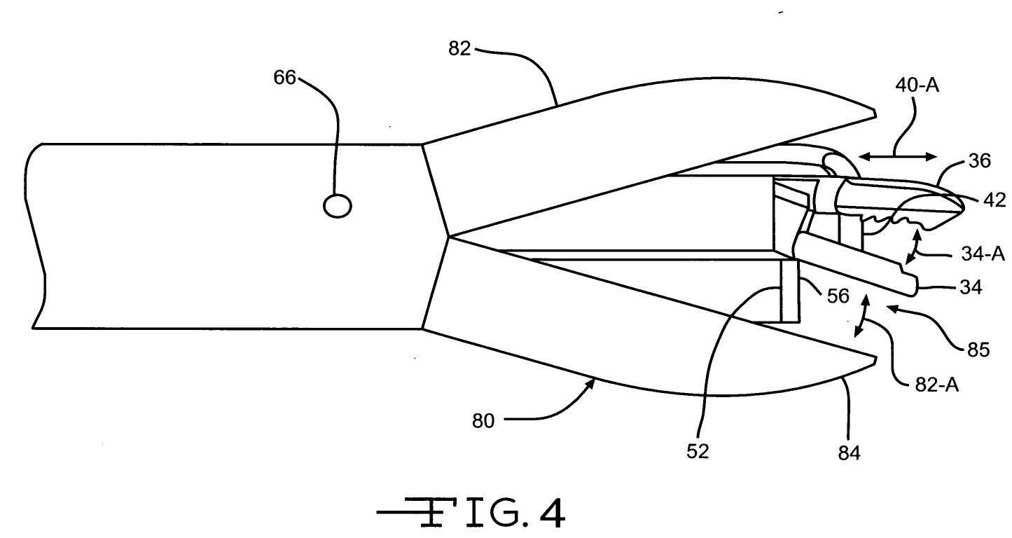

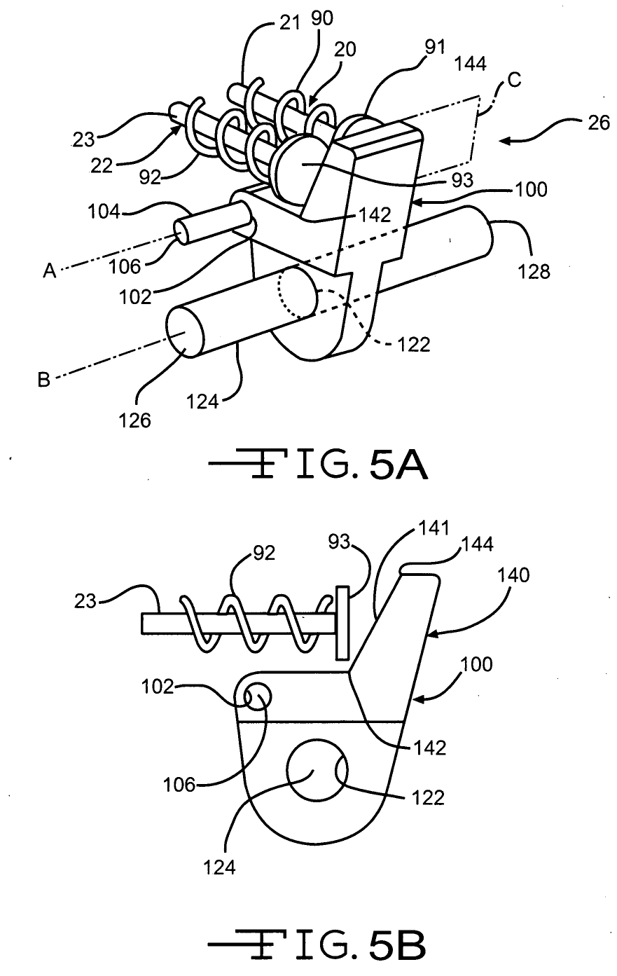

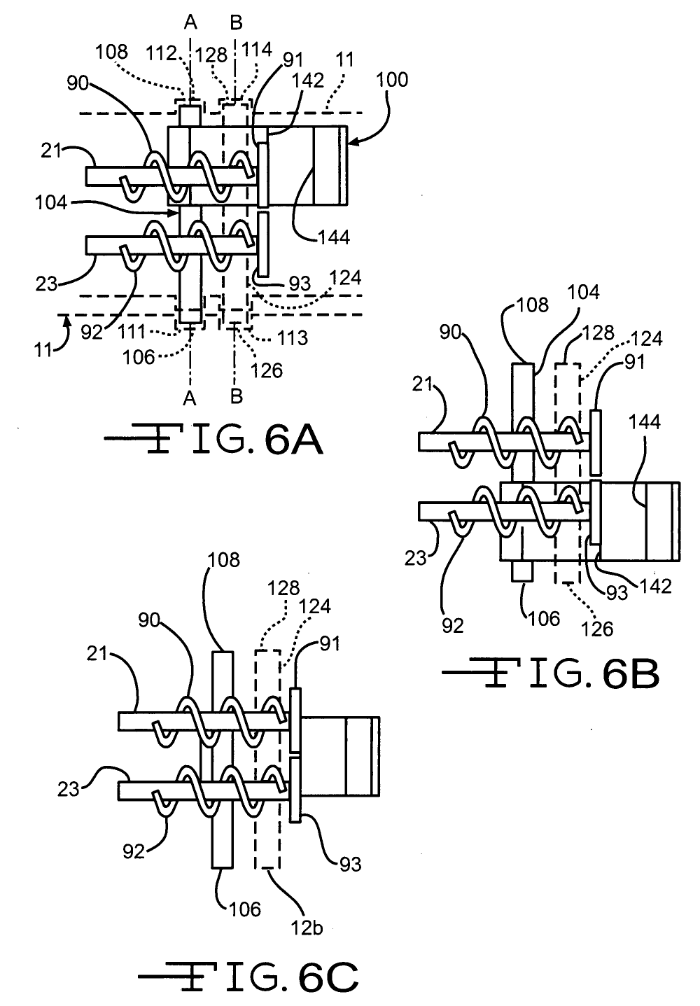

The clip dispensing mechanism 26 includes a movable housing 100 that is operatively mounted in the handle 11. The movable housing 100 has a first opening 102 through which a guide bar 104 extends. As best illustrated in FIG. 6A, the guide bar 104 has axially opposing first and second ends 106 and 108 that are pivotably secured in opposing detents 110 and 112 in the handle 11. The movable housing 100 is slideably movable along a longitudinal axis A that extends through the guide bar 104 between the first and second ends 106 and 108.

The movable housing 100 also has a second opening 122 through which a pivot bar 124 extends. The pivot bar 124 has axially opposing first and second ends 126 and 128 that are held in detents 113 and 114 in the handle 11. The movable housing 100 is slideably movable along a longitudinal axis B that extends through the pivot bar 124 between the first and second ends 126 and 128. The movable housing 100 is also pivotably movable about the axis B in a direction toward the first and second proximal ends 21 and 23 of the clipping devices 20 and 22, as further explained below.

The movable housing 100 is thus: i) slideably movable along the longitudinal axis A extending through the guide bar 104; ii) slideably movable along the longitudinal axis B extending through the pivot bar 124; iii) pivotably movable about the longitudinal axis B through the pivot bar 124; and, iv) can be pivotably movable about its axis.

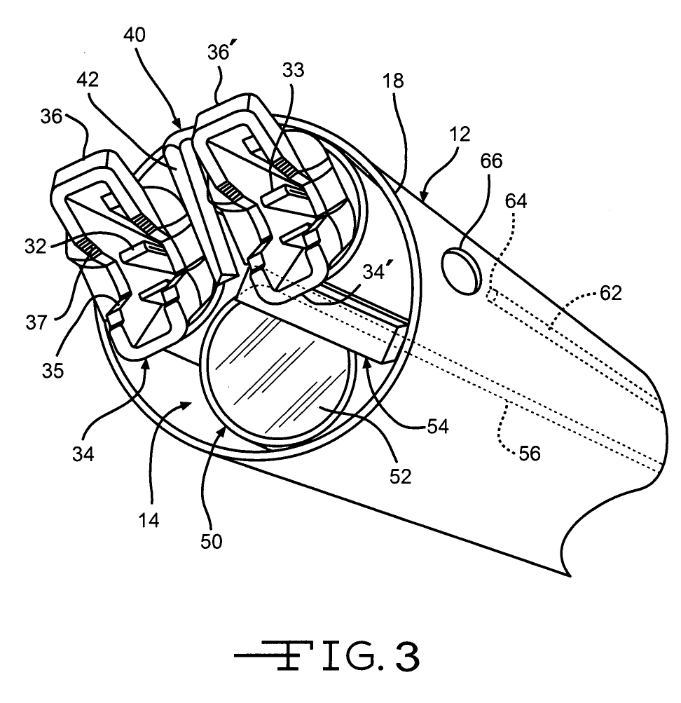

As best shown in FIGS. 5A and 5B, the movable housing 100 includes a hammer 140 having a contact face 141. When not in use, the hammer 140 is in a spaced apart relationship to the first and second proximal ends 21 and 23. The contact face 141 extends in a planar direction C. The plane C, defined by the contact face 141, extends in a direction that is parallel to the axes A and B that extend through the guide bar 104 and the pivot bar 124, respectively. The contact face 141 has a lower, or leading edge 142 and an upper, or trailing, edge 144. The leading edge 142 is positioned at a first distance from the flanges 91 and 93, and the trailing edge 144 is positioned at a second, greater distance from the flanges 91 and 93. As the movable housing 100 is pivoted about the axis A of the pivot bar 124, first the leading edge 142 and then the trailing edge 144 contact at least one of the flanges 91 and/or 93.

In FIGS. 6A and 7A, the movable housing 100 is shown as being positioned or moved to the second end 108 of the guide bar 104 and to the second end 128 of the pivot bar 124. When the movable housing 100 is pivoted about the axis B, the contact face 141 is rotated in a direction toward the first flange 91 of the first clipping device 20. The pivotal rotation of the movable housing 100 causes the leading edge 142 and then the trailing edge 144 of the contact face 141 to contact the first flange 91, thereby compressing the first spring 90. In this manner, the first clipping device 20 is actuated and the surgical clip 32 is dispensed from the clip dispensing mechanism 26 onto the first branch portion B-1.

In FIGS. 6B and 7B, the movable housing 100 is shown as being positioned or moved to the first end 106 of the guide bar 104 and to the first end 126 of the pivot bar 124. When the movable housing 100 is pivoted about the axis B, the hammer 140 is rotated in a direction toward the second flange 93 of the second clipping device 22. The pivotal rotation of the movable housing 100 causes the leading edge 142 and then the trailing edge 144 of the contact face 141 to contact the second flange 93, thereby compressing the second spring 92. In this manner, the second clipping device 22 is actuated and the surgical clip 33 is dispensed from the clip dispensing mechanism 26 onto the second branch portion B-2.

In FIGS. 6C and 7C, the movable housing 100 is moved to a midpoint on the guide bar 104 and to a midpoint on the pivot bar 124. When the movable housing 100 is pivoted about the axis B, the contact face 140 is rotated in a direction toward both the first flange 91 and the second flange 93 on the first and second clipping devices 20 and 22, respectively. The pivotal rotation of the movable housing 100 causes the leading edge 142 and then the trailing edge 144 of the contact face 141 to contact the both the first and second flanges 91 and 93, thereby compressing both the first and second springs 90 and 92, respectively. In this manner, both the first and second clipping devices 20 and 22 are substantially simultaneously activated, and the surgical clips 32 and 33 are dispensed onto the first and second branch portions B-1 and B-2.

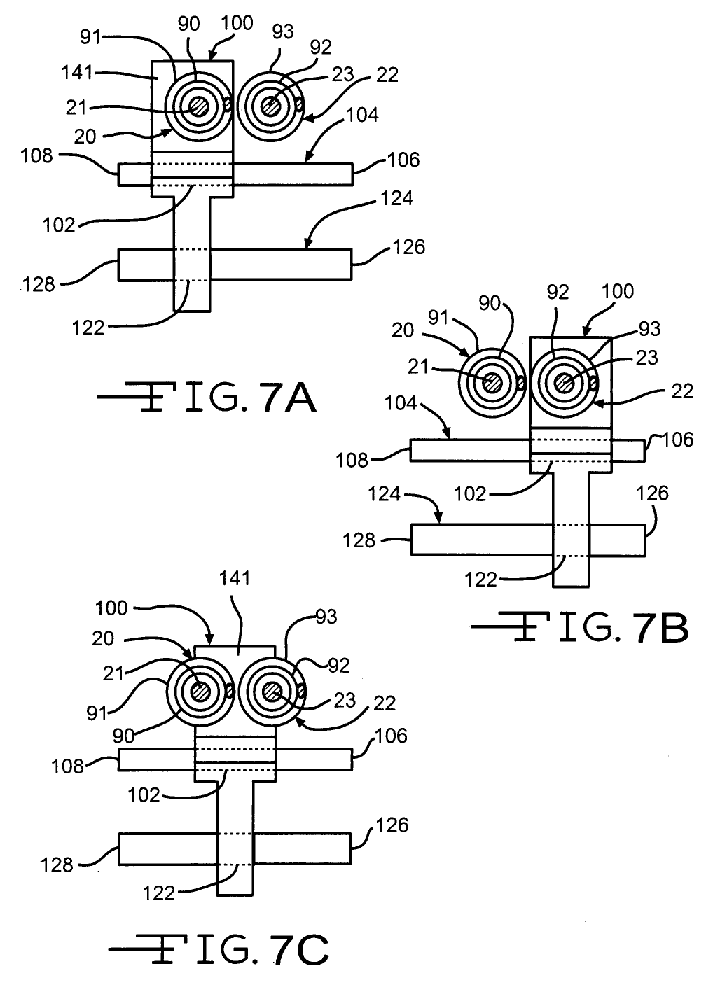

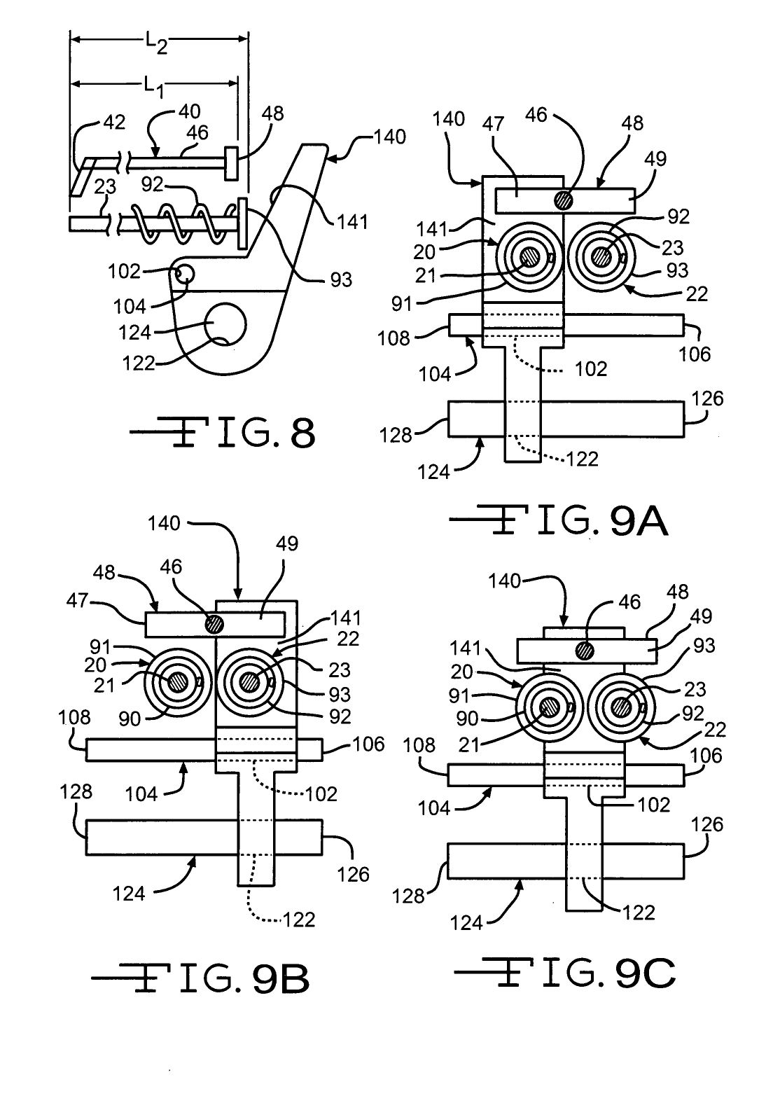

Referring now to FIGS. 8 and 9, the clip dispensing mechanism 26 is shown as also activating the severing device 40. At least the rod 46 of the severing device 40 is in an axial alignment with the first and second cutting devices 20 and 22. In the embodiment shown in FIGS. 8 and 9, the severing device 40 has the cutting tool 42 at a distal end of the rod 46 and further includes a base member 48 at a proximal end of the rod 46. The base member 48 extends radially from the rod 46 such that the base member 48 rod is in a planar relationship with the contact face 141 of the hammer 140. The base member 48 has opposing first and second ends 47 and 49, respectively

In the configuration shown in FIG. 9A, the pivotal rotation of the movable housing 100 will cause the leading edge 142 and then the trailing edge 144 of the contact face 141 to contact the first flange 91 on the first clipping device 20. In this manner, as with the embodiment described above, the first clipping device 20 is activated and the surgical clip 323 is dispensed from the clip dispensing mechanism 26 onto the first branch portion B-1. In this configuration, the pivotal rotation of the movable housing 100 also will cause the contact face 141 to contact at least the first end 47 of the base member 48. The continued rotation of the contact face 141 thus also causes the rod 46 to be axially moved, thereby advancing the cutting tool 42.

Similarly, in the configuration shown in FIG. 9B, the pivotal rotation of the movable housing 100 will cause the leading edge 142 and then the trailing edge 144 of the contact face 141 to contact the second flange 93 on the second clipping device 22. In this manner, as with the embodiment described above, the second clipping device 22 is activated and the surgical clip 33 is dispensed from the clip dispensing mechanism 26 onto the second branch portion B-2. In this configuration, the pivotal rotation of the movable housing 100 also will cause the contact face 141 to contact at least the second end 49 of the base member 48. The pivotal rotation of the contact face 141 thus also causes the rod 46 to be axially moved, thereby advancing the cutting tool 42.

In the illustration in FIG. 9C, the movable housing 100 is moved to a midpoint on the guide bar 104 and to a midpoint on the pivot bar 124. When the movable housing 110 is pivoted about the axis B, the contact face 140 is rotated in a direction toward both the first flange 91 and the second flange 93, thereby compressing both the first and second springs 90 and 92, respectively. In this manner both the first and second clipping devices 20 and 22 are substantially simultaneously activated, and the surgical clips 32 and 33 are dispensed onto the first and second branch portions B-1 and B-2. The pivotal rotation of the movable housing 100 also causes the contact face 141 to contact the base member 48. In this manner, both of the first and second clipping devices 20 and 22 are activated to dispense the first and second surgical clip 32 and 33 substantially simultaneously. Again, the pivotal rotation of the movable housing 100 also will cause the contact face 141 to contact the base member 48. The pivotal rotation of the contact face 141 thus also causes the rod 46 to be axially moved, thereby advancing the cutting tool 42.

It is to be understood, that in certain embodiments, the base member 48 is contacted with the same actuating stroke as for the dispensing of the surgical clips 32 and 33. It should be noted that the actuating stroke can be accomplished in two or more steps: the first part of the stroke closes the clipping devices 20 and 22 to grasp the branch, the second part of the stroke applies the clips 32 and 33; and the third part of the stroke makes the cut. The actuating stroke can be controlled by the clinician so that the sequence can be interrupted and started over again, if needed.

Referring again to FIG. 8, the rod 46 is shown as having a first length L₁ and the clipping devices 20 and 22 are shown as having a second length L₂. The length L₁ of the rod 46 can be the same, shorter or longer than the length L₂ of the clipping devices 20 and 22. The length L₁ of the rod 46, at least in part, determines when the contacting face 141 will strike the base member 48. As such, the multi-function device 10 can be configured to, first, have the clipping devices 20 and 22 dispense the surgical clips 32 and 33; and, second, have the severing device 40 be extended to sever the branch.

It is also to be understood that while the various descriptions herein are directed to use of the multi-function device 10 while being inserted into an opening in a patient, the multi-function device 10 can also be used after the vessel is harvested to clip any branch stubs on the vessel. Also, the multi-function device 10 can be used in an open procedure in addition to the endoscopic-types fully explained herein. In addition, it is to be understood that while the present description herein has been directed to the dissecting and harvesting of a vessel from a patient, that the multi-function device 10 is also useful in many other types of procedures in addition to the exemplary ones described herein.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof. Therefore, it is intended that the invention not be limited to the particular embodiment disclosed herein contemplated for carrying out this invention, but that the invention will include all embodiments falling within the scope of the claims.

What is claimed is:

1. A multi-function device for clipping and harvesting a vessel and any branches extending therefrom comprising:
a sheath configured to be at least partially inserted in a body through a cut skin portion;
a vision system positioned within the sheath;
first and second clipping devices positioned within the sheath, each clipping device configured to advance a surgical clip and to close the advanced surgical clip around the vessel or branch; and,
a severing device positioned within the sheath and configured to sever the vessel or branch.

2. The multi-function device of claim 1, wherein the severing device is interposed between the first and second clipping devices and configured to sever the vessel or branch between the surgical clips.

3. The multi-function device of claim 1, wherein the first and second clipping devices are in a parallel relationship within the sheath.

4. The multi-function device of claim 1, wherein the first clipping device, the second clipping device and the severing device are in a parallel alignment within the sheath.

5. The multi-function device of claim 1, wherein the severing device is configured to be extendible in an axial direction away from the first and second clipping devices.

6. The multi-function device of claim 1, wherein the sheath includes a tip in an axial position over a distal end of the sheath, the tip being configured to be movable between an open position and a closed position, wherein the first and second clipping devices are in an exposed position when the tip is in the open position.

7. The multi-function device of claim 6, wherein the tip has a distal end configured to dissect the vessel from surrounding tissue.

8. The multi-function device of claim 1, further including an insufflation device configured to supply a gas subcutaneously to an area adjacent to the vessel or branch.

9. The multi-function device of claim 1, wherein the severing device comprises one or more of a knife, a bovie, an ultrasonic cauterizing tool, or a bipolar electrocautery tool.

10. The multi-function device of claim 1, further including a clip dispensing mechanism having a movable housing configured to engage at least one of the first or second clipping devices.

11. The multi-function device of claim 10, wherein the movable housing includes a first opening through which a guide bar extends, and a second opening through which a pivot bar extends, wherein the movable housing is configured to be:
i) slidably movable along a longitudinal axis A extending through the guide bar;
ii) slidably movable along a longitudinal axis B extending through the pivot bar;
iii) pivotably movable about the longitudinal axis B through the pivot bar; and,
iii) pivotably movable about the longitudinal axis a through the pivot bar.

12. The multi-function device of claim 10, wherein the movable housing includes a contact face that is in a spaced apart relationship to the first and second clipping devices when the multi-function device is unengaged.

13. The multi-function device of claim 10, wherein the movable housing is configured to substantially simultaneously activate both the first and second clipping devices.

14. The multi-function device of claim 10, wherein the movable housing is configured to: substantially activate: i) the first clipping device followed by the severing device; ii) the second clipping device followed by the severing device; or, iii) the first clipping device and the second clipping device followed by the severing device.

15. A method of clipping branches and/or vessels during harvesting from a body by a multi-function device, the multi-function device including
a sheath configured to be at least partially inserted in a body through a cut skin portion;
first and second clipping devices positioned within the sheath, each clipping device configured to advance a surgical clip and to close the surgical clip around opposing portions of the vessel or branch; and,
a severing device positioned within the sheath and configured to sever the vessel or branch,
wherein the method comprises:
inserting a distal end of the multi-function device into the body alongside the vessel to form a cavity substantially surrounding the vessel or branch;
activating the first and second clipping devices to seal the vessel or branch; and
moving the severing device to a position substantially adjacent to the clipped vessel or branch, and activating the severing device, whereby the clipped vessel or branch is severed.

16. The method of claim 15, including
advancing the first and second clipping devices to a grasping position adjacent to the vessel or branch to be dissected, and thereafter
closing at least a distal portion of the first and second clipping devices on the vessel or branch.

17. The method of claim 15, wherein the first and second clipping devices are actuated substantially simultaneously.

18. The method of claim 15, wherein either the first or the second clipping devices are actuated sequentially.

19. The method of claim 15, wherein the multi-function device further includes a light source and an imaging system, and the method further includes:
activating the light source to at least illuminate a portion of the vessel or branch; and
activating the imaging system to view at least a portion of the illuminated branch;

20. The method of claim 15, wherein the multi-function device further includes an insufflation device, the method further including:
supplying a gas subcutaneously to an area adjacent to the vessel to be dissected and harvested.
